(19) United States
(12) Patent Application Publication
(10) Pub. No.: US 2022/0153868 A1
(43) Pub. Date: May 19, 2022

(54) HDM2 ANTIBODY FOR USE IN TREATING CANCER

(71) Applicant: ONCOLYZE, INC, New York, NY (US)

(72) Inventor: Steven Evans, New York, NY (US)

(21) Appl. No.: 17/432,563

(22) PCT Filed: Feb. 18, 2020
(86) PCT No.: PCT/US20/18552

§ 371 Date: Aug. 20, 2021

Related U.S. Application Data
(60) Provisional application No. 62/808,073, filed on Feb. 20, 2019.

(51) Int. Cl. C07K 16/32; A61P 35/00

(52) U.S. Cl. CPC C07K 16/32; A61P 35/00; C07K 2317/21; A61K 2039/505; C07K 2317/732; C07K 2317/734; C07K 2317/24

(57) ABSTRACT

The invention provides an antibody or antibody fragment selective for HDM-2, an HDM-2 splice variant, or fragment thereof. The invention also provides a method of treating cancer, said method consisting of, consisting essentially of, or comprising administering to a subject in need thereof a therapeutic amount of an antibody or antibody fragment that is selective for membrane bound HDM-2, and any splice variants thereof.

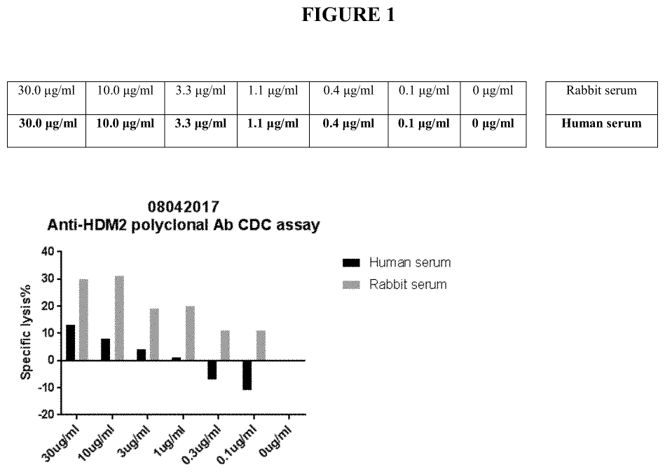

### CROSS-REFERENCE TO RELATED APPLICATION

This application claims priority to U.S. Provisional Application Ser. No. 62/808,073, filed on Feb. 20, 2019, the contents of which are herein incorporated by reference in its entirety.

### BACKGROUND

Cancer treatments which target the p53 protein within the cancer cells have been developed. However, some types of cancer cells do not have p53, while others exhibit p53 in a mutated, and/or inactive form. Thus, these p53 targeting cancer treatments are limited since they do not cause cell death in these types of cancer cells. Thus, p53-targeting cancer treatments are ineffective at treating various types of cancer. Effective cancer treatments remain elusive.

Therefore, there remains a need for improved methods to treat cancer.

### SUMMARY OF THE INVENTION

In one aspect, the invention provides an antibody or antibody fragment selective for HDM-2, an HDM-2 splice variant, or fragment thereof.

In one aspect, the invention provides a method of treating cancer, said method consisting of, consisting essentially of, or comprising administering to a subject in need thereof a therapeutic amount of an antibody or antibody fragment that is selective for membrane bound HDM-2, and any splice variants thereof.

In one aspect, the present invention provides a method of treating cancer in a subject in need thereof, consisting of, consisting essentially of, or comprising administering to the subject a therapeutically effective amount of an antibody or antibody fragment that is selective for HDM-2 or fragment thereof, wherein the cancer includes acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

### DETAILED DESCRIPTION

Provided herein are methods and compositions for the treatment of cancer, including administering to a subject in need thereof an antibody selective for HDM-2, splice variants thereof, and fragments thereof.

As used herein, the term “antibody” refers to any of a polyclonal antibody, a monoclonal antibody, humanized antibodies, non-human species-specific antibodies, synthetic antibodies, single-chain antibodies, chimeric antibodies, human antibodies, affinity matured antibodies, bispecific antibodies, as well as fragments of such molecules that comprise at least one complementarity-determining region.

The antibody may be a Camelid single domain antibody, or portions thereof. In one embodiment, Camelid single-domain antibodies include heavy-chain antibodies found in camelids, or VHH antibody. A VHH antibody of camelid (for example, camel, dromedary, llama, and alpaca) refers to a variable fragment of a camelid single-chain antibody (See Nguyen et al, 2001; Muyldermans, 2001), and also includes an isolated VHH antibody of camelid, a recombinant VHH antibody of camelid, or a synthetic VHH antibody of camelid.

For example, the antibody fragment includes scFv, sdAb, di-scFv, and tri-scFv. scFv is a single-chain variable fragment. sdAb is a single domain antibody. scFv includes the VH and VL domains of an antibody and is connected by a linker. di-scFv includes two scFv molecules connect by a linker. tri-scFv includes three scFv molecules connect by linkers.

The antibody is selective for the surface exposed portions of HDM-2. In one embodiment, the antibody is selective for the p53 binding site of HDM-2. In one embodiment, the antibody is selective for residues 1-109 of HDM-2, 1-50 of HDM-2, 25-75 of HDM-2, 50-109 of HDM-2, 1-491 of HDM-2, or portions thereof. In one embodiment, the antibody is selective for an HDM-2 splice variant.

Gene transcriptional variants resulting from alternative mRNA splicing have been found in many tumor suppressors and oncogenes. Alternative mRNA splicing is sometimes the result of exon skipping. Therefore, mRNA splicing may result in no expression of protein or expression of a protein smaller than the WT protein (a truncated protein). Splice variants of HDM-2 may have a molecular weight of 30-90 kDa, 10-50 kDa, 50-90 kDa, or 75-110 kDa.

In some embodiments, splice variants of HDM-2 have a minimum molecular weight of about 1 kDa, 5, kDa, 10 kDa, 15 kDa, 20 kDa, 25 kDa, 30 kDa, 40 kDa, 50 kDa, 60 kDa, or 75 kDa.

In an embodiment, splice variants of HDM-2 have a maximum molecular weight of about 30 kDa, 35 kDa, 40 kDa, 45 kDa, 50 kDa, 60 kDa, 75 kDa, 80 kDa, 90 kDa, 95 kDa, 100 kDa, or 110 kDa.

Examples of HDM-2 splice variants include MDM2-A, MDM2-B, MDM2-C, MDM2-D, MDM2-E, MDM2-A1, MDM2-KB2, MDM2-KB3, MDM2-JN1, MDM2-DS2, MDM2-DS3, MDM2-IS1, MDM2-GK1, MDM2-PM2, MDM2-EU2, MDM2-BL, MDM2-N, MDM2-FB25, MDM2-FB26, MDM2-FB28, MDM2-FB29, MDM2-FB30, MDM2-FB55, MDM2-281 BP, MDM2-219 BP, MDM2-254 BP, MDM2-F, MDM2-G, MDM2-H, MDM2-LN229A, MDM2-LN229B, MDM2-LN18, MDM2-G116, MDM2-G150, MDM2-VAR2, MDM2-VAR1, MDM2-DELF, MDM2-DELE, AND MDM2-FB60. (See Rosso M., Okoro D. E., Bargonetti J. (2014) Splice Variants of MDM2 in Oncogenesis. In: Deb S., Deb S. (eds) Mutant p53 and MDM2 in Cancer. Subcellular Biochemistry, vol 85. Springer, Dordrecht; the contents of which are incorporated by reference).

In one embodiment, the antibodies described herein include an antibody or antibody fragment that is selective for at least a first target and a second target, wherein the first target and the second target are different. In this embodiment, the antibody may be selective for a first target, a second target, and a third target. The three targets may be three different epitopes on the same target antigen protein, three different epitopes on three different target antigen proteins, or two different epitopes on a first target antigen protein and one epitope on a second target antigen protein.

In one embodiment, the antibodies described herein include one or more of an antibody or antibody fragment that is selective for a first target and a second target, wherein the first target and the second target are different. These antibodies are also referred to as bispecific antibodies. The two targets may be two different epitopes on the same target antigen protein or two different epitopes on two different target antigen proteins. The first target includes HDM2, and the second target is selected from the group consisting of: CD3 [to recruit cytotoxic t-cells], CD16 (FcγRIII) [to recruit other effector cells for ADCC], CD56 [to recruit NK cells], and CD28.

Bispecific antibodies may be derived from the antigen-binding sites, or fragments thereof, of two different antibodies.

Bispecific antibodies can also be generated by the assembly of antibodies with unmodified heavy chain constant regions, such as by the heterodimerization of heavy chains from two different antibodies or homodimerization of heavy chains extended by an additional binding site.

Bispecific antibodies can also be generated by using heavy chains modified to force heterodimerization (e.g., using a knobs-into-holes strategy).

Alternatively, two different antibody fragments, such as scFv, can be fused to a non-immunoglobulin protein, such as albumin, or by way of a chemical linker, such as PEG. Furthermore, two antigen-binding fragments can be directly fused, resulting in small bispecific antibodies molecules. Bispecific antibodies can also be generated by chemical conjugation of two different antibodies.

Bispecific antibodies can also be generated by chemical conjugation of two different whole antibodies.

In one embodiment, this invention provides for a bispecific antibody and use thereof, wherein the first target includes residues 1-109 of HDM-2, 1-50 of HDM-2, 25-75 of HDM-2, 50-109 of HDM-2, 1-491 of HDM-2, or portions thereof; and the second target includes CD3, CD16 (FcγRIII), CD56, or CD28.

In one embodiment, this invention provides for a bispecific antibody and use thereof, wherein the first target includes an HDM-2 splice variant (as described above); and the second target includes CD3, CD16 (FcγRIII), CD56, or CD28.

In one embodiment, the antibodies disclosed herein include an Fc region, or portions of an Fc region. In one embodiment, the antibodies disclosed herein do not include an Fc region, or portions of an Fc region.

In one embodiment, the antibodies of the present invention include monoclonal antibodies.

The antibodies and antibody fragments of the present invention are not conjugated to a pore forming agent, a toxin, a drug, a chemotherapeutic, or a radionuclide.

In one embodiment, the method of the present invention includes co-administration of a chemotherapeutic agent that is not conjugated to the antibody.

In one embodiment, the method of the present invention includes co-administration of an immune system modulator. In one embodiment, the antibody includes an immune system modulator. Examples of immune system modulators include Fc receptor binding domain and C1 complex binding domain.

In another embodiment, the methods of the present invention may further include the step of determining whether the compositions or methods of the present invention causes reduction in the cancer by diagnostic imaging, including MRI, PET scan, and X-Ray.

In another embodiment, the methods of the present invention may further include the step of determining whether the compositions of the present invention causes necrosis, causes membranolysis, or causes poration of cancer cells by measuring the level of serum biomarkers. In one embodiment, the serum biomarker is LDH, cytokeratin, or HDM-2. The level of serum biomarker is measured pre- and post-administration of the compositions disclosed herein. An increase in the serum biomarker between pre- and post-treatment is indicative of cancer cell necrosis, membranolysis, or poration. In one embodiment, the increase is more than 2 times greater, more than 3 times greater, or more than 5 times greater.

In one embodiment, the antibodies described herein are toxic to cancer cells by way of antibody-dependent cell-mediated cytotoxicity (ADCC). ADCC is the process by which antibodies bind a target cell and recruit effector cells to induce target cell death via non-phagocytic mechanisms. In order for an effector cell to carry out ADCC it must express Fc receptors (FcR) that will bind the antibody. The known classes of FcR include FcγR, which bind IgG; FcαR, which bind IgA; and FcεR, which bind IgE. FcγR are the most important for tumor cell clearance by myeloid cells and are comprised of activating FcγRI (CD64), FcγRIIA (CD32A), FcγRIIIA (CD16A), and inhibitory FcγRIIB (CD32B) receptors. Once the FcγR binds antibody it triggers receptor cross-linking and downstream signal propagation. Activating FcγR signal via their immunoreceptor tyrosine-based activation motifs while inhibitory FcγR signal via their immunoreceptor tyrosine-based inhibitory motifs.

In one embodiment, the antibodies disclosed herein include enriched ADCC functionality. In this embodiment, the antibodies include at least one Fc receptor binding domain. The Fc receptor binding domain include FcγRI binding domain (CD64), FcγRIIA (CD32) binding domain, FcγRIIIA (CD16a) binding domain, FcγRIIIB (CD16b) binding domain, Fc√RI binding domain, FcεRII (CD23) binding domain, FcαRI (CD89) binding domain, Fcα/μR binding domain, or FcRn binding domain.

In another embodiment, the methods of the present invention may further include the step of determining whether the compositions or methods of the present invention causes ADCC, and the level of ADCC. This determining step may be helpful to measure the antibody quality, predicting patient responses, and effectiveness of the treatment. Fluorometric techniques may be used to assess the level of ADCC. For example, target cells may be labeled with a fluorescent dye to allow for sensitive determination of cytotoxicity via flow cytometry. As cells are killed they release lactate dehydrogenase and other proteases that can be quantified by supplying fluorogenic substrates in order to accurately assess cytotoxicity without the need to perform any labeling or manipulation of target cells. Impedance-based analysis may also be used to provide a quantitative measure of cell-mediated cytotoxicity that can continuously measure ADCC over time. Peripheral blood mononuclear cells and purified NK cells from donors can be used to quantify ADCC. Clinically available blood samples can be used without isolating NK cells in a rapid ADCC report assay that could be applied to cancer patients to determine the effectiveness of treatment.

In one embodiment, the antibodies described herein are toxic to cancer cells by way of complement-dependent cytotoxicity (CDC). In complement-dependent cytotoxicity (CDC), the C1q binds the antibody and this binding triggers the complement cascade which leads to the formation of the membrane attack complex (MAC) (C5b to C9) at the surface of the target cell, as a result of the classical pathway complement activation. The level of CDC effector function is high for human IgG1 and IgG3, low for IgG2, and null for IgG4.

In one embodiment, the antibodies of the present invention include enriched CDC activity. In this embodiment, the antibodies include at least one C1 complex binding domain.

In another embodiment, the methods of the present invention may further include the step of determining whether the compositions or methods of the present invention causes CDC, and the level of CDC. This determining step may be helpful to measure the antibody quality, predicting patient responses, and effectiveness of the treatment. A complement-dependent cytotoxicity assay can be used to measure and quantitate CDC. For example, a target expressing cell is incubated with an antibody in the presence of human serum containing either active or heat-inactivated complement, and cell lysis is measured.

In another embodiment, the antibodies include one or more Fc receptor binding domains and one or more C1 complex binding domains.

In one embodiment, the methods described herein include administration of an antibody that is enriched for ADCC activity and an antibody that is enriched for CDC activity to a subject in need thereof.

In one embodiment, the methods described herein include co-administration of a peptide having an HDM-2 binding component and a membrane resident component along with the antibody or antibody fragment. The HDM-2 binding component is bound to the membrane resident component.

### Definitions

As used herein, the terms “selective for”, “selective binding”, “specific binding”, or “specifically binding”, in the context of an antibody binding to a target, refers to binding of an Fab, monovalent portion, or target binding portion of that antibody to an target antigen (e.g. HDM-2) with an affinity constant (Kd) of less than about 1×10⁻⁶ M (molar). When an antibody is selective for HDM-2, it specifically binds HDM-2. In certain embodiments, an antibody that exhibits specific binding to an antigen will have a Kd of less than about 1×10⁻⁷ M, less than about 1×10⁻⁸ M, less than about 1×10⁻⁹ M, less than about 1×10⁻¹⁰ M, less than about 1×10⁻¹² M, less than about 1×10⁻¹³ M, less than about 1×10⁻¹⁴ M, less than about 1×10⁻¹⁵ M, or less than about 1×10⁻¹⁸ M.

An antibody or fragment, variant, or derivative thereof is said to competitively inhibit binding of a reference antibody or antigen binding fragment thereof to a given epitope if it binds to that epitope and blocks, to some degree, binding of the reference antibody or antigen binding fragment to the epitope. Competitive inhibition can be determined by any method known in the art including, but not limited to, competition ELISA assays. In certain embodiments, an antibody can be said to competitively inhibit binding of the reference antibody to a given epitope if the antibody reduces binding of the reference antibody by at least 90%, at least 80%, at least 70%, at least 60%, or at least 50% when the antibody is provided at equimolar or higher concentration relative to the reference antibody.

As used herein, the phrase “affinity matured antibody” refers to an antibody comprising one or more alterations in one or more hypervariable regions (HVRs) that provide for an improvement in the affinity of the antibody for an antigen in comparison to an unaltered parent antibody that lacks those alteration(s). Methods for obtaining affinity matured antibodies include, but are not limited to, techniques described in Marks et al. Bio/Technology 10:779-783 (1992), Barbas et al., Proc Nat. Acad. Sci, USA 91:3809-3813 (1994); Schier et al., Gene 169:147-155 (1995); Yelton et al., J. Immunol. 155:1994-2004 (1995); Jackson et al., J. Immunol. 154(7):3310-9 (1995); and Hawkins et al., J. Mol. Biol. 226:889-896 (1992).

As used herein, the term “treat”, when used in the context of a cancer or cell-proliferative disease, refers to any delay in the progression of symptoms associated with a given cancer or cell-proliferative disease in comparison to a subject that has not received the agent.

As used herein, the phrase “therapeutically effective dose” refers to a dose of an agent that provides for an improvement in either the onset of symptoms or the progression of symptoms associated with a given disease in comparison to a subject that has not received the agent.

Throughout this specification, quantities are defined by ranges having a lower boundary and upper boundary, and by lower or upper boundaries. Each lower boundary can be combined with each upper boundary to define a range. Two lower boundary values can be combined to define a range, and two upper boundary values can be combined to define a range. The lower and upper boundaries should each be taken as a separate element.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures, embodiments, or characteristics may be combined in any suitable combinations and/or sub-combinations in one or more embodiments or examples.

### EXAMPLES

### Example 1

### Production, Characterization and Humanization of Anti-HDM-2 Monoclonal Antibody.

Cells expressing HDM-2 or fragments thereof are produced, and harvested with phosphate buffered saline (PBS) containing 25 mM EDTA and used to immunize BALB/c mice. The mice are given injections i.p. of ˜100 cells in 0.5 ml PBS on weeks 0, 2, 5, and 7. The mice with antisera that immunoprecipitated 32P-labeled HDM-2 or fragments thereof are given i.p. injections of a wheat germ agglutinin-Sepharose (WGA) purified HDM-2 membrane extract on weeks 9 and 13. This is followed by an i.v. injection of 0.1 ml of the HDM-2 preparation and the splenocytes are fused with mouse myeloma line X63-Ag8.653. Hybridoma supernatants are screened for HDM-2-binding by ELISA and radioimmunoprecipitation.

The murine monoclonal antibody is humanized, using a “gene conversion mutagenesis” strategy, as described in U.S. Pat. No. 5,821,337, the entire disclosure of which is hereby expressly incorporated by reference.

### Example 2

### Antibody Phage Display Production, Characterization, and Humanization of Anti-HDM-2 Antibody.

Phage display technology is coupled with a cell-based panning strategy.
Cells are transfected and express HDM-2, a splice variant thereof, or a fragment thereof.
Phage libraries are panned against confluent attached monolayers or against cells in suspension.
Phage library sources—immune libraries from blood samples of immunized human donors; universal or single-pot libraries; synthetic universal libraries based on randomized CDR regions inserted into fully synthetic framework sequences; semi-synthetic libraries having a limited number of naïve variable regions of the heavy and light chain are used to insert randomized synthetic CDRs. Naïve universal IgM antibody libraries, wherein the antibody-encoding genes are obtained from of B cells of a large number of donors and from various ethnic backgrounds, ensuring broad coverage of the world's allelic diversity. In one example, the HAL9/10 human naïve antibody gene library is used.

Selection of the peptide ligand is conducted over 4-10 rounds of biopanning. During each round, cells are incubated with a mixture of phage displaying different peptides. Phage that do not bind or bind only to the surface of the cell are washed away. Phage that bind to the cell and are internalized by the cell are retained. These cell-internalized phage are amplified in bacteria, isolated, and used as the input in the next round of biopanning. In each round of selection, the diversity of the phage sample is reduced while the proportion of phage displaying a peptide that mediates cell-specific binding is increased.

Non-specific binding events are reduced by use of blocking agents, such as Bovine Serum Albumin (BSA), milk, or casein. Cell lines expressing the same target antigen may be used to help eliminate any irrelevant binders. Alternatively, or in addition, panning between latex beads coated with the purified form of the antigen and cells expressing the same antigen are alternated between rounds.

Later rounds of panning are performed in the presence of increasing concentration of peptides from Table 1. Panning in the presence of the peptides from Table 1 will increase affinity of the peptides for the membrane bound HDM-2 target.

Incubation time for biopanning steps are performed at 4° C. for 2-16 hours. The incubations may also be performed at 20° C. or 37° C. for 1-20 hours.

Phage recovery may be done with at least one of the following conditions: low pH, alkaline conditions, non-denaturing detergents (NP-40 or Tween20), or direct infection of cell-bound phage with logarithmically growing E. coli.

Optionally, phage display is used in combination with light chain shuffling to exploit the entire theoretical combinatorial repertoire of 10¹⁸ different heavy/light chain combinations available from the human antibody gene to provide the best affinity and kinetic properties. Light-chain shuffling combines the heavy chains of a panned subset of antibodies with the whole light-chain repertoire of the naïve library. These sub-libraries are then re-screened under modified conditions to obtain antibodies with advanced properties such as cross-species reactivity or higher affinity.

The antibodies are tested for their ability to induce CDC and/or ADCC.

Isolated clones are used to make monoclonal antibodies.

TABLE 1
Soluble competitor peptides

| SEQ ID NO: | HDM-2 TARGETING COMPONENT |
|---|---|
| 1 | PPLSQETFSDLWKLL |
| 2 | ETFSDLWKLL |
| 3 | MPRFMDYWEGLN |
| 4 | VQNFIDYWTQQF |
| 5 | TGPAFTHYWATF |
| 6 | IDRAPTFRDHWFALV |
| 7 | PRPALVFADYWETLY |
| 8 | PAFSRFWSDLSAGAH |
| 9 | PXFXDYWXXL |
| 10 | QPTFSDYWKLLP |
| 11 | PPL--TSFXEYWALLX-P |
| 12 | PPLSQTSFAEYWNLL |
| 13 | LTFEHYWAQLTS |
| 14 | TSFAEYWNLLSP |
| 15 | QETFSDLWKLLP |
| 16 | MPRFMDYWEGLN |
| 17 | QQMHLMSYAPGP |
| 18 | TIRPSTTMDSPT |
| 19 | YANPQMEKAFES |
| 20 | LTFEHYWAQLTS |
| 21 | LPNLTWALMPGA |
| 22 | YANPQMEKAFAS |
| 23 | LTFEHYWAQLTS |
| 24 | LLADTTHHRPWT |

### Example 3

CDC Assay with Anti-HDM2 Antibody.

Anti-human/mouse/rat HDM2 polyclonal antibody (AF1244-SP. R&D System) was incubated with MV4-11 cells in the presence of human serum or rabbit serum. The human serum was isolated from a healthy human donor. The rabbit serum was isolated from a rabbit immunized with irrelevant antigen. MV4-11 cells (human, leukemia, acute monocytic).

The principle of CDC assay is: the antibody-antigen immune complex on the cell membrane surface can trigger the classical pathway of the complement system by activation of the C1-Complex (C1qrs) and induce chain reaction for the formation of MAC (membrane attack complex) which causes the cell swells and bursts.

60 μl antibody at 3-fold serial dilution starting from 30 μg/ml were mixed with 40 μl serum, the mixture was then added to 100 μl MV4-11 cells (2×10⁵/m1) in 48-well plate. After 24 hours incubation at 37° C., the cell viability was check by flow cytometry. We record counts of living cells (DAPI⁻), and calculate the lysis rate: % Specific lysis=(Cells only−Cells+Ab)/Cells only ×100%.

The CDC induced cytotoxicity is measured by the cell viability of the tested sample divided by that of the control (no antibody added). The cell lysis is antibody-dose dependent. The rabbit serum could induce a maximum specific lysis of ˜30% when 30 ug/ml anti-HDM2 antibody was added. The human serum induces cell lysis to a lesser extent.

### Example 4

CDC Assay with HDM2 Antibody.

CDC assay is conducted on HDM2 antibody and the following cancer cells: acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

### Example 5

ADCC Assay with HDM2 Antibody.

Anti-human/mouse/rat HDM2 polyclonal antibody (AF1244-SP. R&D System) is incubated with MV4-11 cells in the presence of effector cells expressing Fc receptor CD16. Effector include PBMCs (peripheral blood mononuclear cell) or purified NK cells.

Cell lines for the following cancer cells are tested acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

Cell lines for the aforementioned cancers are well known by those of ordinary skill in the art. For Example, Ca127 is a well known cell line for Head and Neck cancers; OE33 is a well known cell line for Esophageal cancers; SKNAS is a well known cell line for Neuroblastoma; HEPG2 is a well known cell line for Liver cancers; UT7 is a well known cell line for AML; and A172 is a well known cell line for Glioblastoma.

### Example 6

### FACS Analysis to Determine Extracellular Expression of HDM2.

Cells are incubated with no antibody, normal rabbit IgG (isotype control, Santa Cruz Biotechnology, Inc. item #sc-3888) or rabbit polyclonal anti-MDM2/HDM2 (clone N-20, Santa Cruz Biotechnology, Inc. item #sc-813) for 15 minutes in the dark at 4° C. Cells are then washed with 2.0 mL PBS, supernatant aspirated and 150 μL of PBS added to each tube. All cells are then incubated with a secondary goat anti-rabbit IgG conjugated to Phycoerythrin (Santa Cruz Biotechnology, Inc. item #sc-3739) for 15 minutes in the dark at 4° C. Cells are washed with 2.0 mL PBS, supernatant aspirated and 350 μL of PBS added to each tube. Samples are immediately analyzed on a BD FACSCalibur® 4-color flow cytometer using BD CellQuest Pro® software for data acquisition and analysis.

The following conditions are tested for each cell line: 2° Goat anti-Rabbit IgG PE, Rabbit normal IgG with 2° Goat anti-Rabbit IgG PE, and Anti MDM2/HDM2 with 2° Goat anti-Rabbit IgG PE.

The following cells are tested: acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

### Example 7

### In Vivo Analysis of HDM-2 Antibody Activity.

Nu/Nu mice (Harlan Laboratories, Indianapolis, Ind., n=10) weighing 20-22 g, are xenotransplanted subcutaneously (s.c.) with live cancer cells. Tumors are allowed to develop and grow. The following live cancer cells are tested: acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

After tumor formation has occurred, the mice are separated into three groups. One group of mice receives HDM-2 antibody and the other group of mice receives a non-HDM-2 antibody, a control antibody. A third group of mice does not receive antibody. The antibodies are injected into the mice. A description of dosage protocols is listed below. Antibody is administered over the course of 1-14 days.

Since the animals are Nu/Nu mice and, thus, immuno-compromised they are highly susceptible when exposed to pathogens. Surgery and all preceding and post-surgical treatments are therefore performed in a sterile hood.

Alternatively, using the same methodology as described above, live cancer cells described above (1×10⁶ cells/mouse) are transplanted to the peritoneal cavity of a group of mice and the compositions disclosed herein are injected in the right shoulder region at the same time of tumor cell transplantation.

### Example 8

Specific cell surface recognition of membrane bound HDM-2 antigen by the HDM-2 mAb in a panel of cancer cell lines.

HDM-2 antibody is tested for binding to HDM-2 antigen at the plasma membrane of several cancer cell lines by flow cytometry.

The following cancer cells are tested: acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

SEQUENCES
HDM-2 protein sequence
UniProtKB-Q00987 (MDM2_HUMAN)
MCNTNMSVPT DGAVTTSQIP ASEQETLVRP KPLLLKLLKS
VGAQKDTYTM KEVLFYLGQY IMTKRLYDEK QQHIVYCSND
LLGDLFGVPS FSVKEHRKIY TMIYRNLVVV NQQESSDSGT
SVSENRCHLE GGSDQKDLVQ ELQEEKPSSS HLVSRPSTSS
RRRAISETEE NSDELSGERQ RKRHKSDSIS LSFDESLALC
VIREICCERS SSSESTGTPS NPDLDAGVSE HSGDWLDQDS
VSDQFSVEFE VESLDSEDYS LSEEGQELSD EDDEVYQVTV
YQAGESDTDS FEEDPEISLA DYWKCTSCNE MNPPLPSHCN
RCWALRENWL PEDKGKDKGE ISEKAKLENS TQAEEGFDVP
DCKKTIVNDS RESCVEENDD KITQASQSQE SEDYSQPSTS
SSIIYSSQED VKEFEREETQ DKEESVESSL PLNAIEPCVI
CQGRPKNGCI VHGKTGHLMA CFTCAKKLKK RNKPCPVCRQ
PIQMIVLTYF P
PNC-27
PPLSQETFSDLWKLLKKWKMRRNQFWVKVQRG
PNC-28
ETFSDLWKLLKKWKMRRNQFWVKVQRG

1. Method of treating cancer, said method comprising, consisting essentially of, or consisting of administering to a subject in need thereof a therapeutic amount of an antibody or antibody fragment selective for HDM-2.

2. The method according to claim 1, wherein said antibody or antibody fragment is selective for a p53 binding site of HDM-2.

3. The method according to claim 1, wherein said antibody or antibody fragment is selective for residues 1-109 of HDM-2, 1-50 of HDM-2, 25-75 of HDM-2, 50-109 of HDM-2, 1-491 of HDM-2, or portions thereof.

4. The method according to claim 1, wherein said antibody or antibody fragment is selective for membrane bound HDM-2, and any splice variants thereof.

5. The method according to claim 1, wherein said antibody or antibody fragment has a KD value of less than 1×10⁻⁷M, less than 1×10⁻⁸ M, less than 1×10⁻⁹ M, less than 1×10⁻¹⁰ M, less than 1×10⁻¹² M, less than 1×10⁻¹³ M, less than 1×10⁻¹⁵ M, or less than 1×10⁻¹⁸ M.

6. The method according to claim 1, wherein said antibody or antibody fragment is not conjugated to a chemotherapeutic agent.

7. The method according to claim 1, wherein said antibody or antibody fragment is not conjugated to a pore forming agent.

8. The method according to claim 1, wherein said antibody or antibody fragment is not conjugated to a toxin.

9. The method according to claim 1, wherein said antibody or antibody fragment is not conjugated to a radionuclide.

10. The method according to claim 1, wherein said antibody or antibody fragment comprises a modulator of immune effector function.

11. The method according to claim 10, wherein said modulator of immune effector function includes at least one Fc receptor binding domain.

12. The method according to claim 11, wherein the Fc receptor binding domain comprises FcγRI binding domain (CD64), FcγRIIA (CD32) binding domain, FcγRIIIA (CD16a) binding domain, FcγRIIIB (CD16b) binding domain, FεcRI binding domain, FεcRII (CD23) binding domain, FcαRI (CD89) binding domain, Fcα/μR binding domain, or FcRn binding domain.

13. The method according to claim 10, wherein said modulator of immune effector function includes at least one C1 complex binding domain.

14. The method according to claim 1, wherein said antibody or antibody fragment is selective for a first target protein and a second target protein, wherein the first target protein and the second target protein are different.

15. The method according to claim 14, wherein the first target comprises HDM2, and the second target is selected from the group consisting of: CD3, CD16 (FcγRIII), CD56, and CD28.

16. The method according to claim 1, wherein said antibody or antibody fragment is co-administered with a chemotherapeutic agent.

17. The method according to claim 1, wherein cancer is selected from the group consisting of is selected from the group consisting of: acute myeloid leukemia (AML), chronic lymphocytic leukemia (CLL), chronic myeloid leukemia (CML), Multiple Myeloma, Bile duct, Biliary, Myelodysplastic Syndrome, Polycythemia Vera, Childhood leukemia, Monocytic Leukemia, Histiocytic Lymphoma, Promyelocytic Leukemia, Leukemia Stem Cells, Neuroendocrine, Glioblastoma, Astrocytoma, Retinoblastoma, Neuroblastoma, Sarcoma, Uterine cancer, Germ Cell tumor/cancer, Testicular cancer, Wilms tumor, Renal Cell cancer, Mesothelioma, Liposarcoma, Fibrosarcoma, Fibrous Histiocytoma, Ewings Sarcoma, Burkitts/ALL-BCell, T cell ALL, Non Hodgkins lymphoma, Mantle Cell Lymphoma, Thyroid, Bladder, Head and Neck, Esophageal, Liver, Peritoneal carcinomatosis, Pleural Carcinomatosis, Adrenal, gastrointestinal stromal tumors (GIST), Epidermoid, Plasma Cell, T cell Lymphoma cutaneous, breast cancer, lung cancer, pancreatic cancer, melanoma, colon, colon stem cells, leukemia stem cells, ovarian, prostate, and cervical cancer.

18. The method according to claim 1, said method further comprising administering PNC-27 or PNC28.


SEQUENCE LISTING (27 sequences)

SEQ ID NO: 1 - protein, 15 aa, Artificial Sequence
  synthetic sequence
PPLSQETFSDLWKLL

SEQ ID NO: 2 - protein, 10 aa, Artificial Sequence
  synthetic sequence
ETFSDLWKLL

SEQ ID NO: 3 - protein, 12 aa, Artificial Sequence
  synthetic sequence
MPRFMDYWEGLN

SEQ ID NO: 4 - protein, 12 aa, Artificial Sequence
  synthetic sequence
VQNFIDYWTQQF

SEQ ID NO: 5 - protein, 12 aa, Artificial Sequence
  synthetic sequence
TGPAFTHYWATF

SEQ ID NO: 6 - protein, 15 aa, Artificial Sequence
  synthetic sequence
IDRAPTFRDHWFALV

SEQ ID NO: 7 - protein, 15 aa, Artificial Sequence
  synthetic sequence
PRPALVFADYWETLY

SEQ ID NO: 8 - protein, 15 aa, Artificial Sequence
  synthetic sequence
PAFSRFWSDLSAGAH

SEQ ID NO: 9 - protein, 10 aa, Artificial Sequence
  synthetic sequence
  misc_feature (2)..(2): Xaa can be any naturally occurring amino acid
  misc_feature (4)..(4): Xaa can be any naturally occurring amino acid
  misc_feature (8)..(9): Xaa can be any naturally occurring amino acid
PXFXDYWXXL

SEQ ID NO: 10 - protein, 12 aa, Artificial Sequence
  synthetic sequence
QPTFSDYWKLLP

SEQ ID NO: 11 - protein, 15 aa, Artificial Sequence
  synthetic sequence
  misc_feature (7)..(7): Xaa can be any naturally occurring amino acid
  misc_feature (14)..(14): Xaa can be any naturally occurring amino acid
PPLTSFXEYWALLXP

SEQ ID NO: 12 - protein, 15 aa, Artificial Sequence
  synthetic sequence
PPLSQTSFAEYWNLL

SEQ ID NO: 13 - protein, 12 aa, Artificial Sequence
  synthetic sequence
LTFEHYWAQLTS

SEQ ID NO: 14 - protein, 12 aa, Artificial Sequence
  synthetic sequence
TSFAEYWNLLSP

SEQ ID NO: 15 - protein, 12 aa, Artificial Sequence
  synthetic sequence
QETFSDLWKLLP

SEQ ID NO: 16 - protein, 12 aa, Artificial Sequence
  synthetic sequence
MPRFMDYWEGLN

SEQ ID NO: 17 - protein, 12 aa, Artificial Sequence
  synthetic sequence
QQMHLMSYAPGP

SEQ ID NO: 18 - protein, 12 aa, Artificial Sequence
  synthetic sequence
TIRPSTTMDSPT

SEQ ID NO: 19 - protein, 12 aa, Artificial Sequence
  synthetic sequence
YANPQMEKAFES

SEQ ID NO: 20 - protein, 12 aa, Artificial Sequence
  synthetic sequence
LTFEHYWAQLTS

SEQ ID NO: 21 - protein, 12 aa, Artificial Sequence
  synthetic sequence
LPNLTWALMPGA

SEQ ID NO: 22 - protein, 12 aa, Artificial Sequence
  synthetic sequence
YANPQMEKAFAS

SEQ ID NO: 23 - protein, 12 aa, Artificial Sequence
  synthetic sequence
LTFEHYWAQLTS

SEQ ID NO: 24 - protein, 12 aa, Artificial Sequence
  synthetic sequence
LLADTTHHRPWT

SEQ ID NO: 25 - protein, 491 aa, Artificial Sequence
  synthetic sequence
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 26 - protein, 32 aa, Artificial Sequence
  synthetic sequence
PPLSQETFSDLWKLLKKWKMRRNQFWVKVQRG

SEQ ID NO: 27 - protein, 27 aa, Artificial Sequence
  synthetic sequence
ETFSDLWKLLKKWKMRRNQFWVKVQRG